(19) United States
(12) Patent Application Publication
(10) Pub. No.: US 2008/0187510 A1
(43) Pub. Date: Aug. 7, 2008

(54) Methods for Improving Drug Disposition

(76) Inventors: Gian P. Camenisch, Riehen (CH); Hanspeter Nick, Duggingen (CH); Gerhard Gross, Lorrach (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/815,645

(22) PCT Filed: Feb. 8, 2006
(86) PCT No.: PCT/EP2006/001118

§ 371 Date: Aug. 6, 2007

Related U.S. Application Data
(60) Provisional application No. 60/651,684, filed on Feb. 10, 2005.

(51) Int. Cl. A61K 38/19; A61K 31/4196; A61K 31/496; A61P 25/00; A61P 43/00

(52) U.S. Cl. 424/85.1; 514/383; 514/253.01

(57) ABSTRACT

The invention provides a method for improving the bioavailability, preferably, oral bioavailability and/or drug disposition, e.g. brain penetration, of an iron chelator, which method comprises co-administering to a mammal, especially a human, in need of such treatment, a combination of an iron chelator and an efflux protein inhibitor.

The invention provides a method for improving disposition, especially brain penetration, of an iron chelator and its oral bioavailability, e.g. which method comprises co-administering to a mammal, especially a human, in need of such treatment, a combination of an iron chelator and at least one efflux protein inhibitor.

### BACKGROUND OF THE INVENTION

The disposition of many therapeutic agents may be influenced by the action of so-called “efflux pump” proteins which actively eject foreign substances from the cell to give rise, e.g., to the multidrug resistance effect. These drug efflux proteins principally comprise MDR (multidrug resistance protein), MRP (multidrug resistance associated protein) and BCRP (breast cancer resistant protein) type transporters. Some of the best studied efflux proteins include P-glycoprotein (Pgp or MDR1), MRP2 and MXR (BCR-P). These proteins are all expressed, e.g. at the so called blood-brain barrier.

Untreated iron overload can cause severe organ damage, in particular, of the liver, the heart and the endocrine organs, and can lead to death. Newer publications point into the direction that also in brain overload of iron is at least partly involved in diseases like Alzheimer, dementia and Parkinsons. Iron chelators are able to mobilize and excrete the iron deposited in the organs and thus lower the iron-related morbidity and mortality.

### BRIEF SUMMARY OF THE INVENTION

Therefore, one approach to improve drug disposition, especially in brain, is to co-administer at least one efflux protein inhibitor, i.e. a compound that inhibits the function of efflux proteins, with a drug substance. In other words, when at least one efflux protein inhibitor is co-administered with a therapeutic agent which is also a substrate for that specific efflux system, the oral bioavailability and/or the pharmacological active concentrations at the target side (e.g. brain) of the therapeutic agent may be enhanced by inhibiting the efflux mechanism at the various biological membranes/obstacles needed to overcome.

The invention provides a method for improving drug disposition, e.g. brain penetration and/or, the oral bioavailability of an iron chelator, which method comprises co-administering to a mammal, especially a human, in need of such treatment, a combination of an iron chelator and at least one efflux protein inhibitor. The at least one efflux protein inhibitor is administered in an amount such that the bioavailability/disposition of an iron chelator is improved in comparison with what the bioavailability/disposition would be in the absence of the efflux protein inhibitor. The at least one efflux protein inhibitor and an iron chelator are preferably co-administered in an amount such that the combination has a desired therapeutic effect.

The invention provides a method for improving the disposition especially brain uptake and bioavailability of a substituted 3,5-diphenyl-1,2,4-triazole derivative, which method comprises co-administering to a mammal, especially a human, in need of such treatment, a combination of a substituted 3,5-diphenyl-1,2,4-triazole derivative, or a pharmaceutically acceptable salt thereof, and any possible efflux protein inhibitor.

### DETAILED DESCRIPTION OF THE INVENTION

The iron chelators to which the present invention applies are any of those having pharmaceutical utility, e.g. as therapeutic agents for the treatment of diseases which cause an excess of iron in the human or animal or are caused by it.

Iron chelators in combination with at least one efflux protein inhibitor increase the concentration of iron chelators in the brain, which have beneficial effects that mimic hypoxia, including but not limited to, increase expression of enzymes of glycolytic pathways.

Iron chelators in combination with at least one efflux protein inhibitor increase the concentration of iron chelators in the liver, which treat liver metastases, especially when the iron chelators are combined with anti-neoplastic agents.

The term “co-administration” of a combination of an iron chelator, in particular, a substituted 3,5-diphenyl-1,2,4-triazole derivative, and at least one efflux protein inhibitor means that the components can be administered together as a pharmaceutical composition or as part of the same, unitary dosage form. Co-administration also includes administering an iron chelator, in particular, a substituted 3,5-diphenyl-1,2,4-triazole derivative and an efflux protein inhibitor separately but as part of the same therapeutic regimen. The components, if administered separately, need not necessarily be administered at essentially the same time, although they can if so desired. Thus, co-administration includes, e.g., administering an iron chelator, in particular, a substituted 3,5-diphenyl-1,2,4-triazole derivative, plus at least one efflux protein inhibitor as separate dosages or dosage forms, but at the same time. Co-administration also includes separate administration at different times and in any order.

An iron chelator, in particular, a substituted 3,5-diphenyl-1,2,4-triazole derivative, of the present invention may be employed in the form of its pharmaceutically acceptable salts, especially salts with bases, such as appropriate alkali metal or alkaline earth metal salts, e.g., sodium, potassium or magnesium salts; pharmaceutically acceptable transition metal salts, such as zinc salts; or salts with organic amines, such as cyclic amines, such as mono-, di- or tri-lower alkylamines, such as hydroxy-lower alkylamines, e.g. mono-, di- or tri-hydroxy-lower alkylamines, hydroxy-lower alkyl-lower alkylamines or polyhydroxy-lower alkylamines. Cyclic amines are, e.g. morpholine, thiomorpholine, piperidine or pyrrolidine. Suitable mono-lower alkylamines are, e.g. ethyl- and tert-butylamine; di-lower alkylamines are, e.g. diethyl- and di-isopropylamine; and tri-lower alkylamines are, e.g. trimethyl- and triethylamine. Appropriate hydroxy-lower alkylamines are, e.g. mono-, di- and tri-ethanolamine; hydroxy-lower alkyl-lower alkylamines are, e.g. N,N-dimethylamino- and N,N-diethylaminoethanol; a suitable polyhydroxy-lower alkylamine is, e.g. glucosamine. In other cases it is also possible to form acid addition salts, e.g. with strong inorganic acids, such as mineral acids, e.g. sulfuric acid, a phosphoric acid or a hydrohalic acid, with strong organic carboxylic acids, such as lower alkanecarboxylic acids, e.g. acetic acid, such as saturated or unsaturated dicarboxylic acids, e.g. malonic, maleic or fumaric acid or, such as hydroxycarboxylic acids, e.g. tartaric or citric acid, or with sulfonic acids, such as lower alkane- or substituted or unsubstituted benzenesulfonic acids, e.g. methane- or p-toluenesulfonic acid. Compounds of the formula (I), having an acidic group, e.g. carboxyl, and a basic group, e.g. amino, can also be present in the form of internal salts, i.e. in zwitterionic form, or a part of the molecule can be present as an internal salt, and another part as a normal salt.

The term “efflux protein inhibitor”, as used herein, refers to any compound, a pharmaceutical or an excipient compound, that inhibits the action of any ABC transporter, e.g. those disclosed in Bakos et al., Mol Pharmacol, Vol. 57, pp. 760-768 (2002); and Maarten et al., AIDS, Vol. 16, pp. 2295-2301 (2002).

In addition, it may be noted that an efflux protein inhibitor which enhances the bioavailability of an iron chelator may operate by one or more of a variety of mechanisms. That is, as is well-known in the art, it may be a competitive or a non-competitive inhibitor, or it may operate by a mixed mechanism. Whether such an inhibitor can affect the efflux of a certain iron chelator depends, inter alia, upon the relative affinities of the iron chelator and the efflux protein inhibitor; the relative aqueous solubilities of the iron chelator and the efflux protein inhibitor, because this would affect the concentration of the two at the efflux pump in vivo when they are in competition; the absolute aqueous solubility of the efflux protein inhibitor, because it must achieve a sufficient concentration at the efflux pump in vivo to effectively inhibit the efflux; and the dose of the efflux protein inhibitor. For the purpose of this invention, an efflux protein inhibitor is any compound which improves the systemic exposure of an iron chelator, when the iron chelator is dosed orally or by any other route, and which is a substrate and/or an inhibitor of one or more of the drug efflux proteins/activities of the brain and/or blood brain barrier.

As described herein above, the present invention provides a method for improving the bioavailability of iron chelator, in particular, a substituted 3,5-diphenyl-1,2,4-triazole derivative, which method comprises co-administering a combination of an iron chelator and at least one efflux protein inhibitor.

The present invention provides for a combination comprising an iron chelator and at least one efflux protein inhibitor.

The present invention further pertains to the use of a combination comprising an iron chelator and at least one efflux protein inhibitor for the preparation of a medicament to improve the bioavailability of said iron chelator, preferably to the brain.

The present invention pertains to a pharmaceutical composition comprising an iron chelator and at least one efflux protein inhibitor.

Preferably, the at least one efflux protein inhibitor of the present invention is a MDR1, MRP2 and/or MXR inhibitor.

Preferably, 3,5-diphenyl-1,2,4-triazole derivative of the present invention are described in U.S. Pat. No. 6,465,504 B1. The 3,5-diphenyl-1,2,4-triazole derivatives of the present invention have the formula (I)

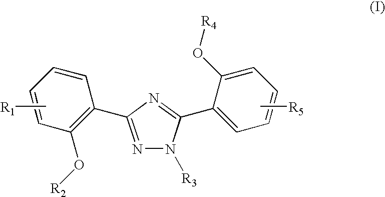

in which
R₁ and R₅, simultaneously or independently of one another, are hydrogen, halogen, hydroxyl, lower alkyl, halo-lower alkyl, lower alkoxy, halo-lower alkoxy, carboxyl, carbamoyl, N-lower alkylcarbamoyl, N,N-di-lower alkylcarbamoyl or nitrile;
R₂ and R₄, simultaneously or independently of one another, are hydrogen, unsubstituted or substituted lower alkanoyl or aroyl, or a radical which can be removed under physiological conditions;
R₃ is hydrogen, lower alkyl, hydroxy-lower alkyl, halo-lower alkyl, carboxy-lower alkyl, lower alkoxycarbonyl-lower alkyl, R₆R₇N—C(O)-lower alkyl, unsubstituted or substituted aryl or aryl-lower alkyl, or unsubstituted or substituted heteroaryl or heteroaralkyl;
R₆ and R₇, simultaneously or independently, of one another are hydrogen, lower alkyl, hydroxy-lower alkyl, alkoxy-lower alkyl, hydroxyalkoxy-lower alkyl, amino-lower alkyl, N-lower alkylamino-lower alkyl, N,N-di-lower alkylamino-lower alkyl, N-(hydroxy-lower alkyl)amino-lower alkyl, N,N-di(hydroxy-lower alkyl)amino-lower alkyl or, together with the nitrogen atom to which they are bonded, form an azaalicyclic ring;
   and salts thereof.

More preferably, a 3,5-diphenyl-1,2,4-triazole derivative of the present invention which is 4-[3,5-bis(2-hydroxyphenyl)-[1,2,4]trizol-1-yl]benzoic acid or a pharmaceutically acceptable salt thereof; is co-administered with a MDR1, MRP2 and/or MXR inhibitor.

As disclosed herein above, an iron chelator, in particular, a 3,5-diphenyl-1,2,4-triazole derivative, and at least one efflux protein inhibitor may be co-administered as a pharmaceutical composition. The components may be administered together in any conventional dosage form, usually also together with a pharmaceutically acceptable carrier or diluent.

For oral administration the pharmaceutical composition comprising an iron chelator, in particular, a 3,5-diphenyl-1,2,4-triazole derivative, and at least one efflux protein inhibitor can take the form of solutions, suspensions, tablets, pills, capsules, powders, microemulsions, unit dose packets and the like. Preferred are tablets and gelatin capsules comprising the active ingredient together with:
a) diluents, e.g. lactose, dextrose, sucrose, mannitol, sorbitol, cellulose and/or glycine;
b) lubricants, e.g. silica, talcum, stearic acid, its magnesium or calcium salt and/or polyethyleneglycol; for tablets, also
c) binders, e.g. magnesium aluminum silicate, starch paste, gelatin, tragacanth, methylcellulose, sodium carboxymethylcellulose and/or polyvinylpyrrolidone; if desired
d) disintegrants, e.g. starches, agar, alginic acid or its sodium salt, or effervescent mixtures; and/or
e) absorbants, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

More specifically, the present invention provides a pharmaceutical composition comprising a therapeutically effective amount of an iron chelator, preferably, a 3,5-diphenyl-1,2,4-triazole derivative, in combination with at least one efflux protein inhibitor, said efflux protein inhibitor being present in an amount such that, following administration, the bioavailability of an iron chelator is statistically significantly improved. In one embodiment, the bioavailability is improved by at least 5%.

Preferably, a pharmaceutical composition of the present invention comprises a MDR1, MRP2 and/or MXR inhibitor.

Preferably, a pharmaceutical composition of the present invention comprises a 3,5-diphenyl-1,2,4-triazole derivative of the formula (I)

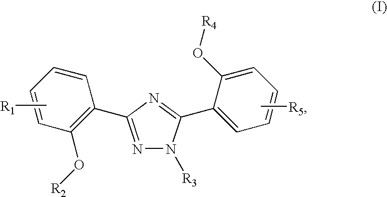

in which
R₁ and R₅, simultaneously or independently of one another, are hydrogen, halogen, hydroxyl, lower alkyl, halo-lower alkyl, lower alkoxy, halo-lower alkoxy, carboxyl, carbamoyl, N-lower alkylcarbamoyl, N,N-di-lower alkylcarbamoyl or nitrile;
R₂ and R₄, simultaneously or independently of one another, are hydrogen, unsubstituted or substituted lower alkanoyl or aroyl, or a radical which can be removed under physiological conditions;
R₃ is hydrogen, lower alkyl, hydroxy-lower alkyl, halo-lower alkyl, carboxy-lower alkyl, lower alkoxycarbonyl-lower alkyl, R₆R₇N—C(O)-lower alkyl, unsubstituted or substituted aryl or aryl-lower alkyl, or unsubstituted or substituted heteroaryl or heteroaralkyl;
R₆ and R₇, simultaneously or independently of one another, are hydrogen, lower alkyl, hydroxy-lower alkyl, alkoxy-lower alkyl, hydroxyalkoxy-lower alkyl, amino-lower alkyl, N-lower alkylamino-lower alkyl, N,N-di-lower alkylamino-lower alkyl, N-(hydroxy-lower alkyl)amino-lower alkyl, N,N-di(hydroxy-lower alkyl)amino-lower alkyl or, together with the nitrogen atom to which they are bonded, form an azaalicyclic ring;
   and salts thereof; in combination with a MDR1, MRP2 and/or MXR inhibitor.

More preferably, a pharmaceutical composition of the present invention comprises a 3,5-diphenyl-1,2,4-triazole derivative which is 4-[3,5-bis(2-hydroxyphenyl)-[1,2,4]trizol-1-yl]benzoic acid (Compound I) or a pharmaceutically acceptable salt thereof in combination with a MDR1, MRP2 and/or MXR (also called BCR-P) inhibitor.

MRP1 inhibitors are leukotriene C4, NEM-GS, probenecid, furosemid, penicillin G, and indomethacin. Preferably, the MRP1 inhibitors according to invention are probenecid, furosemid, penicillin G, and indomethacin.

MDR1 inhibitors are sulfinpyrazone, ritonavir, indinavir, saquinavir.

MRP-2 inhibitors are leukotriene C4, NEM-GS, probenecid, indomethacin, penicillin G, ritonavir, indinavir, saquinavir, furosemide, methotrexate, sulfinpyrazone,

One embodiment of the invention pertains to a combination which comprises a 3,5-diphenyl-1,2,4-triazole derivative which is 4-[3,5-bis(2-hydroxyphenyl)-[1,2,4]trizol-1-yl]benzoic acid (Compound I) or a pharmaceutically acceptable salt thereof in combination with a MDR1 inhibitor selected from the group consisting of sulfinpyrazone, ritonavir, indinavir and saquinavir

In another embodiment, the present invention pertains to the combination which comprises a 3,5-diphenyl-1,2,4-triazole derivative which is 4-[3,5-bis(2-hydroxyphenyl)-[1,2,4]trizol-1-yl]benzoic acid (Compound I) or a pharmaceutically acceptable salt thereof in combination with a MRP-2 inhibitor selected from the group consisting of leukotriene C4, NEM-GS, probenecid, indomethacin, penicillin G, ritonavir, indinavir, saquinavir, furosemide, methotrexate, sulfinpyrazone. Preferably, the present invention pertains to the combination which comprises a 3,5-diphenyl-1,2,4-triazole derivative which is 4-[3,5-bis(2-hydroxyphenyl)-[1,2,4]trizol-1-yl]benzoic acid (Compound I) or a pharmaceutically acceptable salt thereof in combination with a MRP-2 inhibitor selected from the group consisting of probenecid and indomethacin.

In another embodiment, the present invention pertains to the combination which comprises a 3,5-diphenyl-1,2,4-triazole derivative which is 4-[3,5-bis(2-hydroxyphenyl)-[1,2,4]trizol-1-yl]benzoic acid (Compound I) or a pharmaceutically acceptable salt thereof in combination with a MRP-1 inhibitor selected from the group consisting of leukotriene C4, NEM-GS, probenecid, furosemid, penicillin G, and indomethacin.

Preferably, the present invention pertains to the combination which comprises a 3,5-diphenyl-1,2,4-triazole derivative which is 4-[3,5-bis(2-hydroxyphenyl)-[1,2,4]trizol-1-yl]benzoic acid (Compound I) or a pharmaceutically acceptable salt thereof in combination with a MRP-1 inhibitor selected from the group consisting of probenecid, furosemid, penicillin G, and indomethacin.

Preferably, the bioavailability of a iron chelator, in particular, a 3,5-diphenyl-1,2,4-triazole derivative is statistically significantly improved. In one embodiment, the bioavailability is improved by at least 5%.

The blood-brain barrier (BBB) and the blood-CSF barrier (BCSFB) represent the main interfaces between the central nervous system (CNS) and the peripheral circulation. Drug compounds like Compound I that are substrates for ATP transporters such as MDR1, MRP2 and BCRP which are highly expressed in the BBB and BCSFB may very efficiently removed from the CNS, thus limiting brain uptake, by the activity of these efflux systems. Inhibition of one or several of these ATP transporters by an efflux protein inhibitor may improve/increase the exposure of Compound I to the brain.

Bioavailability of a drug may be assessed as known in the art by measuring area under the curves (AUCs), where AUC is plotting the serum or plasma concentration of a drug along the ordinate (Y-axis) against time along the abscissa (X-axis). Generally, the values for AUC represent a number of values taken from all the subjects in a test population and are, therefore, mean values averaged over the entire test population.

Co-administration of iron chelator and at least one efflux protein inhibitor may also increase Cₘₐₓ relative to dosing the iron chelator in the absence of at least one efflux protein inhibitor, and this is provided as a further aspect of the invention. Cₘₐₓ is also well-understood in the art as an abbreviation for the maximum drug concentration in serum or plasma of a test subject.

Since the present invention has an aspect that relates to treatment with a combination of compounds which may be co-administered separately, the invention also relates to combining separate pharmaceutical compositions in kit form. The kit comprises two separate pharmaceutical compositions:
(1) a composition comprising an iron chelator, in particular, a 3,5-diphenyl-1,2,4-triazole derivative, plus a pharmaceutically acceptable carrier or diluent; and
(2) a composition comprising at least one efflux protein inhibitor, plus a pharmaceutically acceptable carrier or diluent.

The amounts of (1) and (2) are such that, when co-administered separately, the brain penetration/bioavailability of an iron chelator, in particular, a 3,5-diphenyl-1,2,4-triazole derivative, is statistically significantly improved. In one embodiment, the bioavailability is improved by at least 5%. The kit comprises a container for containing the separate compositions, such as a divided bottle or a divided foil packet, wherein each compartment contains a plurality of dosage forms, e.g. tablets, comprising (1) or (2). Alternatively, rather than separating the active ingredient-containing dosage forms, the kit may contain separate compartments each of which contains a whole dosage which in turn comprises separate dosage forms. An example of this type of kit is a blister pack wherein each individual blister contains two (or more) tablets, one (or more) tablet(s) comprising a pharmaceutical composition (1), and the second (or more) tablet(s) comprising a pharmaceutical composition (2). Typically the kit comprises directions for the administration of the separate components. The kit form is particularly advantageous when the separate components are preferably administered in different dosage forms, e.g. oral and parenteral, are administered at different dosage intervals, or when, titration of the individual components of the combination is desired by the prescribing physician. In the case of the instant invention a kit therefore comprises:
(1) a therapeutically effective amount of a composition comprising an iron chelator, in particular, a 3,5-diphenyl-1,2,4-triazole derivative, and a pharmaceutically acceptable carrier or diluent, in a first dosage form;
(2) a composition comprising at least one efflux protein inhibitor in an amount such that, following administration, the bioavailability of an iron chelator, in particular, a 3,5-diphenyl-1,2,4-triazole derivative, is statistically significantly improved and a pharmaceutically acceptable carrier or diluent, in a second dosage form; and
(3) a container for containing said first and second dosage forms.

In another embodiment, the present invention relates to a use of at least one efflux protein inhibitor, in particular, a MDR1, MRP2 and/or MXR inhibitor, for the manufacture of a medicament to improve the bioavailability, preferably oral or brain bioavailability, of an iron chelator, preferably, a 3,5-diphenyl-1,2,4-triazole derivative.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way.

The efflux protein(s) involved in the extrusion of a drug substance may be identified, and the corresponding kinetic parameters may be determined, i.e. Michaelis-Menten Constant (Kₘ), Maximal Transporter Activity (Vₘₐₓ) and/or inhibitor concentration needed to cause 50% inhibition of Vₘₐₓ (IC₅₀), using methods known in the art, e.g. by purified membrane vesicles from insect or mammalian cells or selected cell lines expressing high levels of the selected ABC transporter(s).

### EXAMPLE 1

### ATPase Assay

In this assay the ABC transporters remove substrates out of reconstituted cell membranes by using ATP hydrolysis as an energy source. ATP hydrolysis yields inorganic phosphate (Pi), which can be detected by a simple calorimetric reaction. The amount of Pi liberated by the transporter is proportional to the activity of the transporter. Membrane preparations containing ABC transporters show a baseline ATPase activity that varies for different transporters. Transported substrates increase this baseline ATPase activity. As illustrated herein (Table 1), Compound I increases the ATPase activity in reconstituted membranes expressing high levels of BCRP (with a Kₘ value of about 1 μM) or MRP2 (with a Kₘ value of about 10 μM), suggesting Compound I to be actively transported by these transporter proteins. No activation of the MDR1 efflux could be observed.

TABLE 1
ATPase activity in the presence of Compound I in reconstituted
membranes expressing high levels of BCRP, MRP2 or MDR1

ATPase activity (nmol Pi/min/mg protein)

| Compound I (μM) | MDR1 | MRP2 | BCRP |
|---|---|---|---|
| 0.04 | 18.7 ± 0.6 | 5.1 ± 0.6 | 32.9 ± 1.0 |
| 0.13 | 18.0 ± 0.7 | 5.4 ± 0.3 | 35.5 ± 0.8 |
| 0.40 | 17.4 ± 1.3 | 5.4 ± 0.1 | 34.2 ± 8.0 |
| 1.21 | 17.0 ± 1.0 | 5.1 ± 0.4 | 49.4 ± 0.5 |
| 3.63 | 17.3 ± 0.3 | 5.0 ± 0.0 | 61.1 ± 0.3 |
| 10.89 | 17.3 ± 0.5 | 5.2 ± 0.4 | 65.9 ± 0.1 |
| 32.67 | 17.3 ± 0.4 | 6.3 ± 0.2 | 65.7 ± 0.5 |
| 98.0 | 16.7 ± 0.5 | 8.3 ± 0.7 | 60.3 ± 1.6 |
| base line | 50.2 | 16.0 | 65.2 |

### EXAMPLE 2

### Vesicular Uptake Assay

In this assay ATP-dependent uptake into membrane vesicles with inside-out orientation is determined. Interaction of Compound I with ABC transporters is measured indirectly by incubating the purified membrane vesicles with known radioactive probe substrates ([³H]LTC_{4 [}0.2 μM] for MRP2—LTC₄ stands for Leutriene C₄- and [³H]E₁S [0.5 μM]—E₁S stands for estrange sulfate—for BCRP) in the presence and absence (negative control) of different concentrations of Compound I or a well-known positive control compound (Benzbromanone for MRP2 and Sulphasalazine for BCRP). As illustrated herein (Tables 2 and 3), Compound I inhibits [³H]E₁S as well as [³H]LTC₄ transport mediated by BCRP (IC₅₀≅1 μM) and MRP2 (IC₅₀≅50 μM), respectively.

TABLE 2
Effect of Compound I on the vesicular uptake of [³H]E₁S
in isolated membrane vesicles over-expressing BCRP

| | | Vesicular uptake | | | |
|---|---|---|---|---|---|
| | Conc. | ATP activation | | AMP activation | |
| Compound | [μM] | [pmol/mg/min] | SD | [pmol/mg/min] | SD |
| −(Neg Control) | 0 | 36.6 | 3.2 | 23.2 | 1.7 |
| Compound I | 0.1 | 33.2 | 0.3 | 13.8 | 1.3 |
| Compound I | 1 | 25.3 | 2.3 | 13.8 | 1.3 |
| Compound I | 10 | 18.3 | 1.1 | 20.7 | 3.4 |
| Compound I | 100 | n.d. | n.d. | n.d. | n.d. |
| Sulfasalazine | 7500 | 18.2 | 2.5 | 17.9 | 3.2 |
| (Positive | | | | | |

n.d = not determined

TABLE 3
Effect of Compound I on the vesicular uptake of [³H]LTC₄
in isolated membrane vesicles over-expressing MRP2

| | | Vesicular uptake | | | |
|---|---|---|---|---|---|
| | Conc. | ATP activation | | AMP activation | |
| Compound | [μM] | [pmol/mg/min] | SD | [pmol/mg/min] | SD |
| −(Negative | 0 | 30.3 | 0.9 | 5.4 | 0.8 |
| Compound I | 0.1 | n.d. | n.d. | n.d. | n.d. |
| Compound I | 1 | n.d. | n.d. | n.d. | n.d. |
| Compound I | 10 | 25.6 | 1.1 | 5.4 | 0.8 |
| Compound I | 100 | 11.5 | 0.8 | 5.8 | 0.7 |
| Benzbro- | 3000 | 10.8 | 1.0 | 11.1 | 0.4 |
| manone | | | | | |
| (Positive | | | | | |
| Control) | | | | | |

n.d = not determined

### EXAMPLE 3

### Permeability Assay

Alternatively, the in vitro transporter affinity of a drug substance can be determined and approximated by measuring the compound permeability across cells known to express ABC transporters, as e.g. the Caco-2 cell line. Interaction of Compound I with ABC transporter(s) is measured by determining the concentration-dependent compound transport across Caco-2 cell monolayers from the apical (AP) to basolateral (BL) as well as the basolateral to apical side. As illustrated herein (FIG. 4), Compound I is clearly identified as a substrate for one or several prominent efflux system(s). At low Compound I concentrations apical to basolateral transport is significantly lower than basolateral to apical transport. The transport is concentration-dependent and bi-directional permeability values approximately converge at about 50 μM, indicating that complete efflux transporter saturation is achieved at this Compound I concentration (apparent Kₘ≅5 μM).

TABLE 4
Bi-directional transport of Compound
I across Caco-2 cell monolayers

| | | Caco-2 permeability | | | | | |
|---|---|---|---|---|---|---|---|
| | | Pₐₚₚ | | | Pₐₚₚ | | |
| | | _{(AP-BL)} | | | _{(BL-AP)} | | |
| | Conc. | [10⁻⁵ | | | [10⁻⁵ | | |
| Compound | [μM] | cm/min] | SD | | cm/min] | SD | |
| Compound I | 1 | 6.0 | 2.2 | (100) | 93.2 | 5.1 | (112) |
| Compound I | 5 | 46.0 | 21.2 | (91) | 142.7 | 22.2 | (117) |
| Compound I | 10 | 69.8 | 27.9 | (89) | 133.3 | 18.6 | (118) |
| Compound I | 50 | 87.6 | 2.4 | (80) | 128.9 | 8.1 | (122) |

1. A combination comprising (a) an iron chelator and (b) at least one efflux protein inhibitor.

2. The combination according to claim 1 wherein the iron chelator is a 3,5-diphenyl-1,2,4-triazole derivative, or a pharmaceutically acceptable salt thereof.

3. The combination according to claim 2, wherein the at least one efflux protein inhibitor is selected from a MDR1 inhibitor, an MRP2 inhibitor and a MXR inhibitor.

4. The combination according to claim 3, wherein the 3,5-diphenyl-1,2,4-triazole derivative has the formula (I) 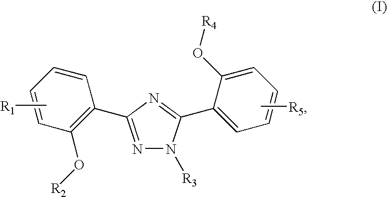
in which
R₁ and R₅, simultaneously or independently of one another, are hydrogen, halogen, hydroxyl, lower alkyl, halo-lower alkyl, lower alkoxy, halo-lower alkoxy, carboxyl, carbamoyl, N-lower alkylcarbamoyl, N,N-di-lower alkylcarbamoyl or nitrile;
R₂ and R₄, simultaneously or independently of one another, are hydrogen, unsubstituted or substituted lower alkanoyl or aroyl, or a radical which can be removed under physiological conditions;
R₃ is hydrogen, lower alkyl, hydroxy-lower alkyl, halo-lower alkyl, carboxy-lower alkyl, lower alkoxycarbonyl-lower alkyl, R₆R₇N—C(O)-lower alkyl, unsubstituted or substituted aryl or aryl-lower alkyl, or unsubstituted or substituted heteroaryl or heteroaralkyl;
R₆ and R₇, simultaneously or independently of one another, are hydrogen, lower alkyl, hydroxy-lower alkyl, alkoxy-lower alkyl, hydroxyalkoxy-lower alkyl, amino-lower alkyl, N-lower alkylamino-lower alkyl, N,N-di-lower alkylamino-lower alkyl, N-(hydroxy-lower alkyl)amino-lower alkyl, N,N-di(hydroxy-lower alkyl)amino-lower alkyl or, together with the nitrogen atom to which they are bonded, form an azaalicyclic ring;
and salts thereof.

5. The combination according to claim 4, wherein the 3,5-diphenyl-1,2,4-triazole derivative is 4-[3,5-bis(2-hydroxyphenyl)-[1,2,4]triazol-1-yl]benzoic acid, or a pharmaceutically acceptable salt thereof.

6. Use of the combination according to claim 1 for the preparation of a medicament for the treatment of diseases caused by brain iron overload.

7. A pharmaceutical composition comprising the combination according to claim 1.

8. The pharmaceutical composition according to claim 7 comprising a therapeutically effective amount of an iron chelator in combination with at least one efflux protein inhibitor, said at least one efflux protein inhibitor being present in an amount such that, following administration, the bioavailability of said iron chelator is improved by at least 5%.

9. A method of treating a brain disease caused by iron overload, which method comprises co-administering, to a mammal in need such treatment, a combination of an iron chelator and at least one efflux protein inhibitor.

10. A method according to claim 9, wherein the iron chelator is a 3,5-diphenyl-1,2,4-triazole derivative, or a pharmaceutically acceptable salt thereof.

11. A method according to claim 10, wherein the at least one efflux protein inhibitor is selected from a MDRI inhibitor, an MRP2 inhibitor and a MXR inhibitor.

12. A method according to claim 11, wherein the 3,5-diphenyl-1,2,4-triazole derivative has the formula (I) 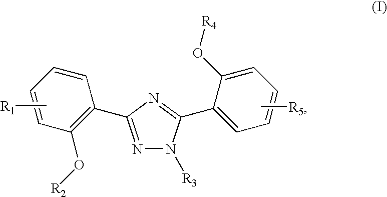
in which
R₁ and R₅, simultaneously or independently of one another, are hydrogen, halogen, hydroxyl, lower alkyl, halo-lower alkyl, lower alkoxy, halo-lower alkoxy, carboxyl, carbamoyl, N-lower alkylcarbamoyl, N,N-di-lower alkylcarbamoyl or nitrile;
R₂ and R₄, simultaneously or independently of one another, are hydrogen, unsubstituted or substituted lower alkanoyl or aroyl, or a radical which can be removed under physiological conditions;
R₃ is hydrogen, lower alkyl, hydroxy-lower alkyl, halo-lower alkyl, carboxy-lower alkyl, lower alkoxycarbonyl-lower alkyl, R₆R₇N—C(O)-lower alkyl, unsubstituted or substituted aryl or aryl-lower alkyl, or unsubstituted or substituted heteroaryl or heteroaralkyl;
R₆ and R₇, simultaneously or independently of one another, are hydrogen, lower alkyl, hydroxy-lower alkyl, alkoxy-lower alkyl, hydroxyalkoxy-lower alkyl, amino-lower alkyl, N-lower alkylamino-lower alkyl, N,N-di-lower alkylamino-lower alkyl, N-(hydroxy-lower alkyl)amino-lower alkyl, N,N-di(hydroxy-lower alkyl)amino-lower alkyl or, together with the nitrogen atom to which they are bonded, form an azaalicyclic ring;
and salts thereof.

13. A method according to claim 12, wherein the 3,5-diphenyl-1,2,4-triazole derivative is 4-[3,5-bis(2-hydroxyphenyl)-[1,2,4]triazol-1-yl]benzoic acid, or a pharmaceutically acceptable salt thereof.
